(19) United States
(12) Patent Application Publication
(10) Pub. No.: US 2008/0133023 A1
(43) Pub. Date: Jun. 5, 2008

(54) PROVISIONAL PROSTHETIC COMPONENT FORMED OF MULTIPLE MATERIALS

(75) Inventors: Ryan D. Schlotterback, Warsaw, IN (US); Barish Banerjee, Warsaw, IN (US); Randy G. Smythe, Tippecanoe, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER TECHNOLOGY, INC., Warsaw, IN (US)

(21) Appl. No.: 11/538,828

(22) Filed: Oct. 5, 2006

(51) Int. Cl. A61F 2/36; A61F 2/30

(52) U.S. Cl. 623/22.42; 623/22.43; 623/22.46; 623/914; 623/20.15

(57) ABSTRACT

The present invention relates to provisional prosthetic components for a medical device and the surgical methods for utilizing the same. In one embodiment, the provisional prosthetic component includes a first portion and a core. The first portion is capable of mating with another prosthetic component. The first portion of the provisional prosthetic component may have a scratch hardness which is less than the scratch hardness of the prosthetic component. This allows for the first portion to be placed in mating engagement with the prosthetic component without scratching or damaging the junction of the prosthetic component. Additionally, the core of the provisional prosthetic component may have a scratch hardness greater than the scratch hardness of the first portion of the provisional prosthetic component and/or a stiffness sufficient to minimize deflection of the provisional prosthetic component during trial reduction and range of motion testing.

### BACKGROUND

1. Field of the Invention

The present invention relates to a provisional prosthetic component for a medical device and the surgical methods for utilizing the same.

2. Description of the Related Art

Prostheses are commonly utilized to repair and/or replace damaged bone and tissue in the human body. For example, a hip prosthesis may be implanted to replace damaged or destroyed bone in the femur and/or acetabulum and recreate the natural, anatomical articulation of the hip joint. To implant a prosthesis, orthopedic surgery is performed which requires the creation of an incision in the skin of the patient and may necessitate the retraction of large portions of surrounding tissue to provide the surgeon with sufficient access to the surgical site. However, some orthopedic surgeries are performed utilizing minimally invasive techniques. When these techniques are utilized, a minimally invasive surgery is performed which may require only a small incision and very limited retraction of surrounding tissue. This may shorten a patient's recovery time and expedite healing.

To facilitate minimally invasive surgery, modular prostheses may be utilized. Modular prostheses have several individual, distinct components which are connected together to form the final, implanted prosthesis. Since the individual components of a modular system are smaller then the prostheses of a non-modular system, less retraction of tissue is needed at the surgical site to provide sufficient working space to the surgeon. Additionally, a modular prosthesis may include different interchangeable components, e.g., several stems having different lengths which are included in a modular femoral prosthesis system. This provides the surgeon greater flexibility to assemble a prosthesis that more closely approximates the patient's anatomy.

In addition to the final, implanted components of a modular prosthesis system, a modular prosthesis system may also include trial or provisional components which replicate the size and shape of the final, implanted components of the modular prosthesis system. The use of provisional components provides the surgeon with the ability to test the ultimate configuration of the prosthesis prior to the implantation of the final components. By trialing, i.e., testing, the surgeon is able to ensure that the fit, alignment, and range of motion provided by the final prosthesis will closely match the patient's natural anatomy.

Additionally, some modular prosthesis systems provide for the implantation of a final component prior to the use of mating provisional components for trialing. In these systems, the provisional components should be designed to accommodate the forces encountered during trial reduction and range of motion testing without compromising the functionality of the mating junction of the final component.

### SUMMARY

The present invention relates to provisional prosthetic components for a medical device and the surgical methods for utilizing the same. In one embodiment, the provisional prosthetic component includes a first portion and a core. The first portion is capable of mating with another modular prosthetic component. The first portion of the provisional prosthetic component may have a scratch hardness which is less than the scratch hardness of the modular prosthetic component. This allows for the first portion to be placed in mating engagement with the prosthetic component without scratching or damaging the junction of the modular prosthetic component. Additionally, the core of the provisional prosthetic component may have a scratch hardness greater than the scratch hardness of the first portion of the provisional prosthetic component and/or a stiffness sufficient to minimize deflection of the provisional prosthetic component during trial reduction and range of motion testing.

Advantageously, the design of the present provisional component allows for the use of a non-provisional modular prosthetic component during trialing of the provisional components. This results from the present provisional prosthetic component providing a connection with the non-provisional modular prosthetic component which will not scratch or damage the junction of the non-provisional modular prosthetic component. Additionally, the present provisional component includes a core which is stiff enough to limit deflection of the present provisional component during trialing to provide an accurate representation of the relative position of the final, non-provisional prosthetic component.

In one form thereof, the present invention provides a provisional prosthetic component, including a body sized to replicate the orientation of at least one component of a modular prosthetic component, the body including a core formed of a core material; and a first portion overlying the core, the first portion having a provisional prosthetic junction surface sized and shaped to mate with a final modular prosthetic junction surface of a component of the modular prosthetic component, the provisional prosthetic junction surface formed from a provisional prosthetic material, the final modular prosthetic junction surface formed from a final modular prosthetic material, the provisional prosthetic material having a lower scratch hardness than the final modular prosthetic material, whereby interaction of the provisional prosthetic material with the final modular prosthetic material will more likely cause scratching of the provisional prosthetic material than the final modular prosthetic material, the core material having a stiffness greater than the provisional prosthetic material from which the provisional prosthetic junction surface is formed.

In another form thereof, the present invention provides a provisional prosthetic component system, including a body sized to replicate the orientation of at least one component of a modular prosthetic component, the body including a core formed of a core material; and a first portion overlying the core, the first portion having a provisional prosthetic junction surface sized and shaped to mate with a final modular prosthetic junction surface of a component of the modular prosthetic component, the provisional prosthetic junction formed from a plastic, the final modular prosthetic junction surface formed from a final modular prosthetic material having a higher scratch hardness than the plastic from which the provisional prosthetic junction surface is formed, whereby interaction of the plastic with the final modular prosthetic material will more likely cause scratching of the plastic than the final modular prosthetic material.

In another form thereof, the present invention provides a method for trialing a prosthetic device including the steps of implanting a component of a modular prosthesis within a joint of a patient's body; attaching a provisional component to the component of the modular prosthesis, the provisional component having a body sized to replicate the orientation of at least one component of the modular prosthetic component, the body including a core formed of a core material; and a first portion overlying the core, the first portion having a provisional prosthetic junction surface sized and shaped to mate with a final modular prosthetic junction surface of the component of a modular prosthesis, the provisional prosthetic junction formed from a provisional material, the final modular prosthetic junction surface formed from a final modular prosthetic material, the provisional prosthetic material having a lower scratch hardness than the final modular prosthetic material, whereby interaction of the provisional prosthetic material with the final modular prosthetic material will more likely cause scratching of the provisional prosthetic material than the final modular prosthetic material, the core material having a stiffness greater than the provisional prosthetic material from which the provisional prosthetic junction surface is formed; reducing the joint, including the provisional component; evaluating the performance of the provisional component; and replacing the provisional component with a corresponding component of the modular prosthesis.

### BRIEF DESCRIPTION OF THE DRAWINGS

The above-mentioned and other features and advantages of this invention, and the manner of attaining them, will become more apparent and the invention itself will be better understood by reference to the following descriptions of embodiments of the invention taken in conjunction with the accompanying drawings, wherein:

FIG. 1 is an exploded perspective view of a modular femoral prosthesis;

FIG. 2 is an exploded perspective view of a provisional component of the present invention;

FIG. 3 is a perspective view of the assembled component of FIG. 2;

FIG. 4 is a side view of the core of a provisional component according to another embodiment;

FIG. 5 is a side view of the core of a provisional component according to another embodiment;

FIG. 6 is a partial perspective view of a provisional component according to another embodiment;

FIG. 7 is a partial cross-section of the provisional component of FIG. 6 taken along line 7-7;

FIG. 8 is a perspective view of the core of a provisional component according to another embodiment;

FIG. 9 is a perspective view of a provisional component according to another embodiment incorporating the core of FIG. 8; and

FIG. 10 is a cross-sectional view of the provisional component of FIG. 9 taken along line 10-10.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

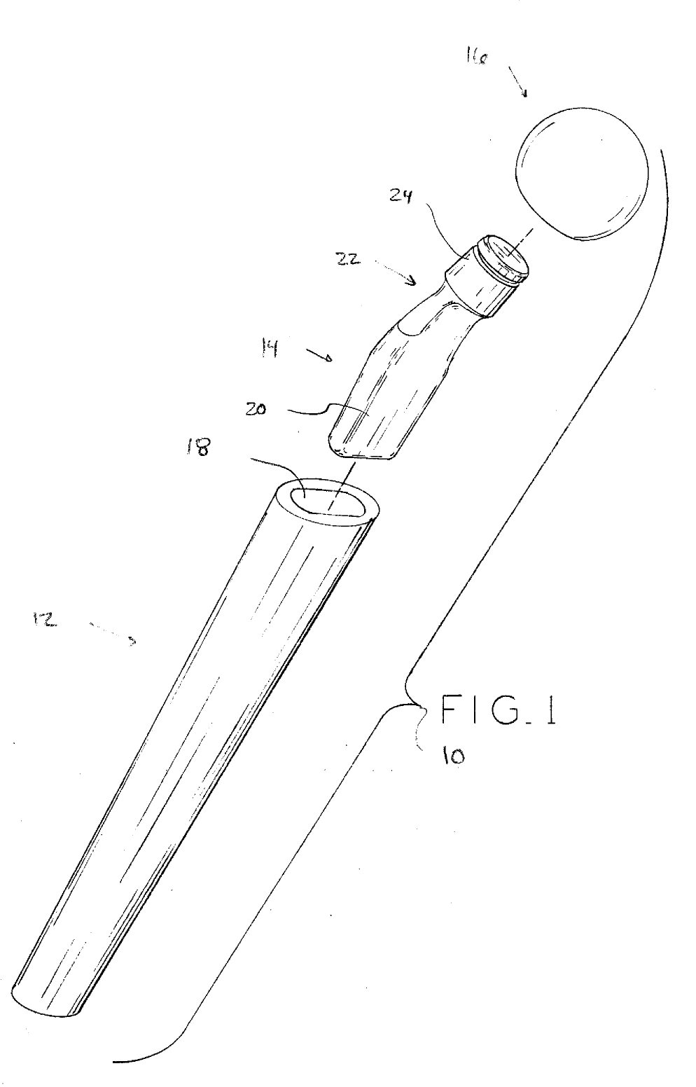

FIG. 1 depicts a modular prosthesis system as modular femoral prosthesis 10. Modular femoral prosthesis 10 includes stem 12, neck 14, and head 16. Stem 12 is designed to seat within the intramedullary canal of a femur and includes female tapered cavity 18. Neck 14 connects to stem 12 via insertion of male tapered portion 20 into female tapered cavity 18. In use, upper portion 22 of neck 14 can extend from the intramedullary canal of the femur into the joint space of the hip. Neck 14 further includes second male taper 24 designed to mate with a female tapered cavity (not shown) within head 16. Head 16 replaces the natural femoral head and, once implanted, articulates against the natural acetabulum or an implanted acetabular prosthesis. To prepare the femur for insertion of stem 12, the upper portion of the femur is, in certain surgical techniques, resected and a series of broaches having an increasing diameter can be utilized to prepare the femur to receive modular femoral prosthesis 10.

As discussed above, provisional components replicate the orientation of the individual components, i.e., stem 12, neck 14, and head 16, of a modular prosthesis component but are designed solely for trialing, i.e., testing, purposes. Referring to FIG. 2, provisional neck 30 includes first portion 32 and core 34. While the provisional prosthetic component of the present invention is depicted and described herein as a provisional prosthetic neck for a modular femoral prosthesis, the design of the provisional prosthetic component of the present invention may further embody additional prosthetic components, e.g., the combination of a provisional femoral neck and provisional femoral head, and is also applicable to other prosthetic devices which may be used in other parts of the human anatomy where the benefits of the present invention would be advantageous, e.g., other long bone prostheses. First portion 32 includes an outer male tapered surface 36 configured for mating engagement with female tapered cavity 18 of non-provisional prosthetic stem 12. Core 34 also includes an outer male tapered surface 38, which is located on provisional head junction 40, for mating with a corresponding cavity in a provisional head (not shown). While the connections between the modular components of the femoral prosthesis system depicted and described herein are shown and discussed as male/female or Morse taper connections, any known mechanism for attachment of module prosthetics may also be used.

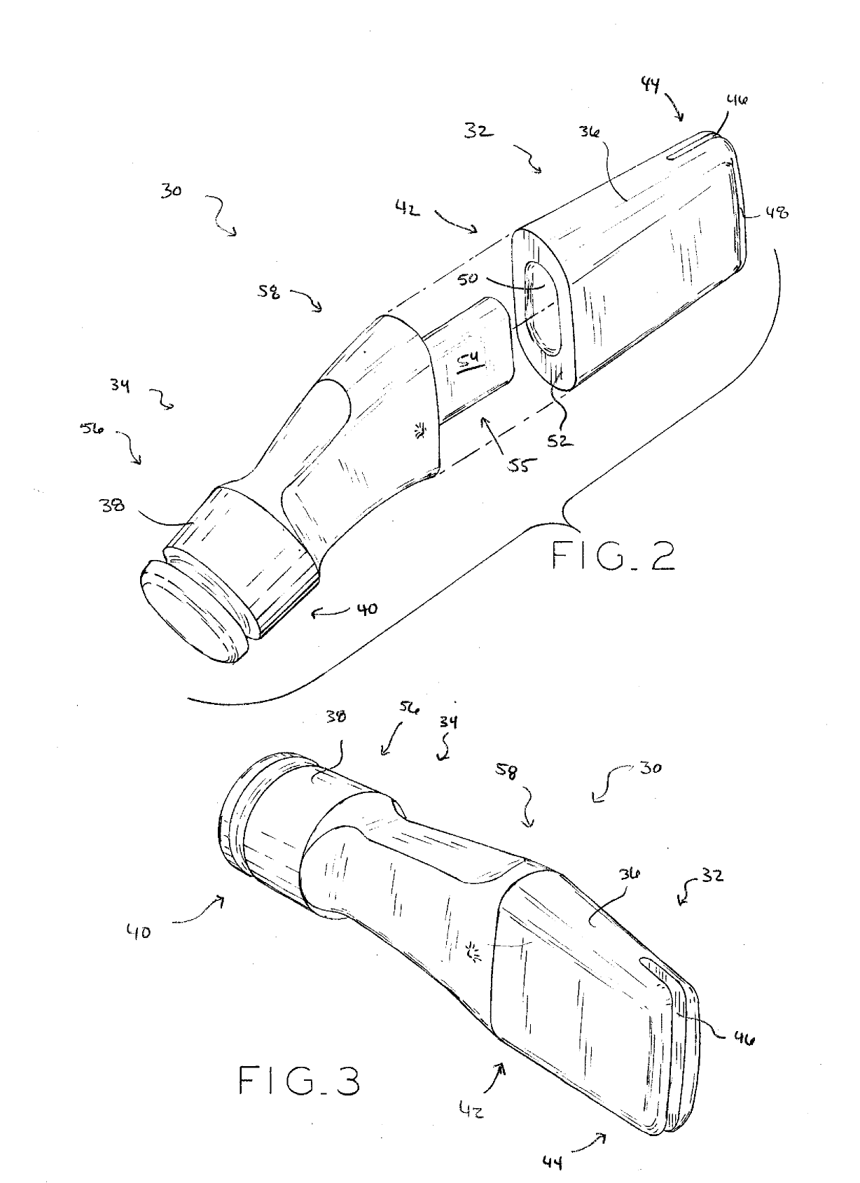

As discussed above, the implantation of the present modular femoral prosthesis system begins by properly preparing the patient's femur for receipt of a femoral prosthesis. Once prepared, non-provisional stem 12, shown in FIG. 1, is implanted within the intramedullary canal of the femur. With non-provisional stem 12 implanted, provisional neck 30 (FIG. 3), which corresponds in orientation to non-provisional neck 14 (FIG. 1), is attached to the stem 12 via engagement of tapered surface 36, shown in FIG. 2, of first portion 32 with female tapered cavity 18 of stem 12. With provisional neck 30 attached to stem 12, a provisional head (not shown), which corresponds in orientation to non-provisional head 16, is attached to male tapered surface 38 of core 34 of provisional neck 30. With the components connected, the provisional head is reduced, placing it in mating articulation with the natural or prosthetic acetabulum.

To ensure that the components provide the proper fit, the patient's leg is subjected to range of motion testing. If, after testing, the surgeon determines that the provisional components are acceptable, then provisional neck 30 and the provisional head (not shown) are removed and replaced by non-provisional neck 14 and non-provisional head 16. Alternatively, if the testing indicates that another non-provisional neck 14 and/or non-provisional head 16 would be more advantageous, provisional neck 30 and/or provisional head (not shown) are removed and replaced with provisional components corresponding in orientation to the newly selected non-provisional neck 14 and/or non-provisional head 16 and the process is repeated. As noted above, the provisional components of the present invention replicate the orientation of the non-provisional components. However, such replication does not require that the provisional components have the same size and shape as the non-provisional components.

In one exemplary embodiment, a plurality of different cores 34 are provided to allow for the exchange of one of the plurality of cores 34 for another of the plurality of cores 34. This would, after the insertion of first portion 32 into non-provisional stem 12, as discussed in detail above, allow the surgeon to exchange cores 34 for additional trialing without needing to remove first portion 32 from non-provisional stem 12.

As set forth above, provisional neck 30 includes first portion 32 and core 34. First portion 32 may be constructed from a material having a scratch hardness that is less than the scratch hardness of non-provisional stem 12. Scratch hardness refers to the resistance of a material to penetration, i.e., scratching, by other materials. Thus, a material having a high scratch hardness can penetrate, i.e., scratch, a material having a lower scratch hardness. Similarly, a material having a lower scratch hardness is less likely to penetrate, i.e., scratch, a material having a higher scratch hardness. By having a lower scratch hardness, first portion 32 is prevented from scratching or damaging the walls defining female tapered cavity 18 of non-provisional stem 12 when inserted therein. For example, when stem 12 is constructed of titanium or a titanium alloy, first portion 32 may be constructed of a suitable plastic having a lower scratch hardness than titanium or titanium alloy, such as carbon fiber polyetheretherketone, polyethelyene, polytetrafluoroethylene, or polyphenylsulfone, such as Radel® A and Radel® B. Radel® is a registered trademark of Amoco Polymers, Inc. of Alpharetta, Ga. In one exemplary embodiment, first portion 32 has a scratch hardness substantially less then the scratch hardness of core 34, for example, when first portion 32 is made of polyethylene and core 34 is made of a cobalt chromium alloy.

Because first portion 32 may be made from a suitable plastic, injection molding and other known forming methods may be used to manufacture first portion 32. Additionally, because numerous sizes and designs of provisional component 30 may be needed for a single modular prosthesis system, the ability to quickly differentiate between the various provisional components is advantageous. Therefore, prior to molding or otherwise forming first portion 32, a coloring may be added to the plastic to color code the varying provisional components. Thus, first portion 32 of the smallest size provisional neck 30 may be a made to have a first color, the next larger sized first portion 32 may be made to have a second color, and so on. This type of standardized color coding system provides the surgeon with a quick and easy way to identify the appropriate provisional component for trialing.

In contrast to the materials used for constructing first portion 32, core 34 may be formed from a material having a scratch hardness greater than the scratch hardness of the material forming first portion 32. This material should be capable of withstanding the loads encountered in the joint during trial reduction and range of motion testing while accurately replicating the positions of the final prosthetic components. Thus, when first portion 32 and core 34 are connected, core 34 may provide greater rigidity or stiffness to provisional neck 30, which limits deflection of provisional neck 30 and helps ensure accurate orientation of the components during trialing. Core 34 may be manufactured from metal, for example, including titanium alloys, cobalt chromium alloys, or surgical grade stainless steel. If core 34 is constructed of metal, traditional forming and machining techniques may be utilized.

As shown in FIG. 2, first portion 32 and core 34 may be manufactured as separate, individual components and thereafter assembled, as shown in FIG. 3. First portion 32 and core 34 may be assembled by the manufacturer or, alternatively, may be assembled by the surgeon before or during the surgical procedure. In an exemplary embodiment, discussed above, a plurality of different cores 34 are provided to allow for the exchange of one of the plurality of cores 34 for another of the plurality of cores 34.

As shown in FIGS. 2 and 3, first portion 32 includes proximal end 42 and distal end 44. Distal end 44 may include slot 46 cut therein to facilitate insertion into and retention of first portion 32 within female tapered cavity 18 of non-provisional stem 12. Additionally, projection 48, shown in FIG. 2, may be positioned substantially near distal end 44 of first portion 32. During insertion of distal end 44 into cavity 18 of stem 12, projection 48 may provide a tactile or audible indication to the surgeon that provisional neck 30 is fully seated, i.e., bottomed out, within female tapered cavity 18 of non-provisional stem 12. Proximal end 44 of first portion 32 includes cavity 50 formed therein and extending from surface 52 towards distal end 44. The walls defining cavity 50 form a female taper configured for mating engagement with male tapered surface 54 of projection 55 of core 34.

Referring to FIGS. 2 and 3, core 34 includes proximal end 56 and distal end 58. Distal end 58 includes projection 55, shown in FIG. 2, with male tapered surface 54 configured for mating engagement with cavity 50 of proximal end 42 of first portion 32. In one exemplary embodiment, epoxy may be placed within cavity 50 of first portion 32 prior to insertion of projection 55 therein to facilitate the securement of first portion 32 and core 34 together. In another exemplary embodiment, provisional prosthesis 30 includes a bore (not shown) extending through first portion 32 and projection 55 of core 34. In this embodiment, the bore is sized to accept a cross-pin (not shown) to secure first portion 32 to core 34.

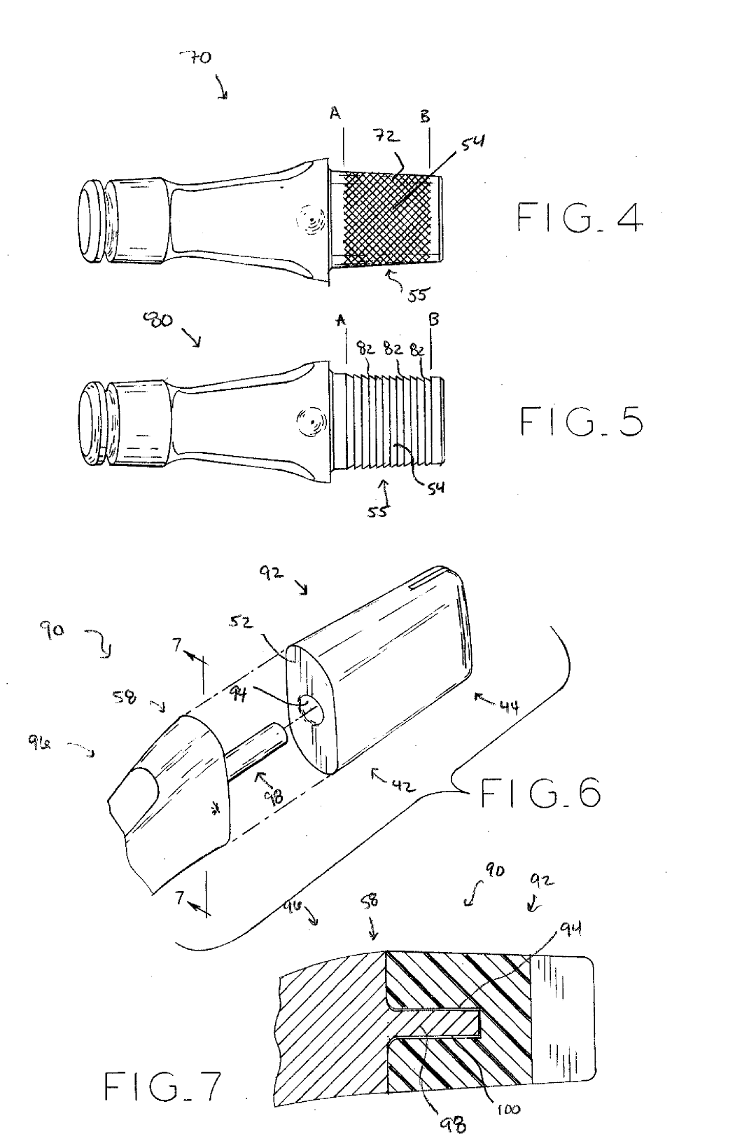

FIGS. 4-10 depict individual cores and assembled provisional necks according to additional embodiments of the present invention. These cores and provisional necks include several features which are identical to the embodiment of FIGS. 2 and 3, discussed above, and identical reference numerals have been used to indicate identical or substantially identical features therebetween.

Referring to FIG. 4, core 70 includes crosshatching 72 on outer surface 54 of projection 55 extending between line A and line B. Crosshatching 72 provides additional frictional engagement between projection 55 of core 70 and the walls of male tapered cavity 50 of first portion 32 to retain first portion 32 and core 70 in mutual engagement. In another exemplary embodiment, shown in FIG. 5, core 80 includes a series of annular teeth 82 on outer surface 54 of projection 55 extending between line A and line B. In a manner similar to crosshatching 72, annular teeth 82 provide additional frictional engagement between projection 55 of core 80 and the walls of male tapered cavity 50 of first portion 32.

FIGS. 6 and 7 depict a portion of provisional neck 90 according to another exemplary embodiment of the present invention. First portion 92 of provisional neck 90 includes cylindrical cavity 94 extending from surface 52 of proximal end 42 toward distal end 44. Additionally, distal end 58 of core 96 includes cylindrical projection 98 sized for receipt within cylindrical cavity 94 of first portion 92. In one exemplary embodiment, epoxy may be placed within cylindrical cavity 94 prior to insertion of cylindrical projection 98 therein to facilitate securement between first portion 32 and core 96. The epoxy may substantially entirely fill space 100, shown in FIG. 7, between the walls defining cylindrical cavity 94 and cylindrical projection 98. Then, once the epoxy has cured, first portion 32 and core 96 are rigidly secured to one another.

FIG. 8 depicts another embodiment of core 32 of provisional neck 30 as core 110. Projection 55 of core 110 includes roughened outer surface 112. For example, roughened outer surface 112 may include a tooth pattern, annular grooves, ribs, ridges, or crosshatching. Roughened outer surface 112 of projection 55 is sized to fit within an oversized cavity of a first portion (not shown). The oversized cavity of the first portion is formed slightly larger than projection 55 of core 110. Once projection 55 is positioned within the oversized cavity of the first portion, energy may be exerted on either core 110 or the first portion, e.g., by heating, causing the first portion to interdigitate with roughened surface 112 of projection 55. This interdigitation provides secure attachment between the first portion and core 110.

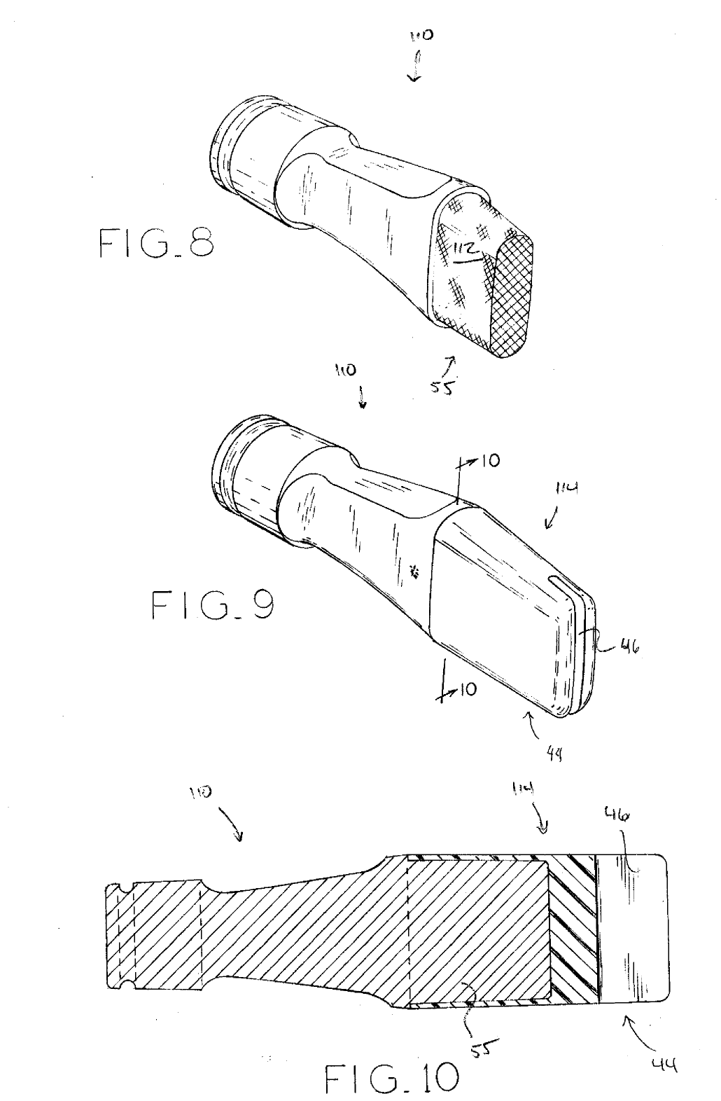

Alternatively, in another exemplary embodiment, projection 55 of core 112, shown in FIG. 8, may be utilized as a substrate or base portion upon which first portion 114 may be formed, as shown in FIGS. 9-10. In this embodiment, roughened outer surface 112 of projection 55 provides an attachment surface for directly forming or overmolding first portion 114 thereon. For example, first portion 114 may be injection molded directly onto projection 55. In the same manner as described in detail above, roughened outer surface 112 of projection 55 provides for secure attachment between first portion 110 and core 114.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A provisional prosthetic component, comprising:
a body sized to replicate an orientation of at least one component of a modular prosthetic component, said body comprising:
a core formed of a core material; and
a first portion overlying said core, said first portion having a provisional prosthetic junction surface sized and shaped to mate with a final modular prosthetic junction surface of a component of the modular prosthetic component, said provisional prosthetic junction surface formed from a provisional prosthetic material, the final modular prosthetic junction surface formed from a final modular prosthetic material, said provisional prosthetic material having a lower scratch hardness than the final modular prosthetic material, whereby interaction of said provisional prosthetic material with the final modular prosthetic material will more likely cause scratching of said provisional prosthetic material than the final modular prosthetic material, said core material having a stiffness greater than said provisional prosthetic material from which said provisional prosthetic junction surface is formed.

2. The provisional prosthetic component of claim 1, wherein said core forms a projection and said first portion includes a cavity sized for receipt of said projection to secure said first portion to said core.

3. The provisional prosthetic component of claim 2, further comprising epoxy positioned within said cavity to further secure said first portion to said core.

4. The provisional prosthetic component of claim 2, wherein said projection forms a male tapered surface and the walls defining said cavity form a female tapered surface.

5. The provisional prosthetic component of claim 1, wherein the scratch hardness of said provisional prosthetic material is substantially lower than the scratch hardness of the final modular prosthetic material.

6. The provisional prosthetic component of claim 5, wherein said body comprises a femoral neck.

7. The provisional prosthetic component of claim 1, wherein said provisional prosthetic material is a plastic.

8. The provisional prosthetic component system of claim 7, wherein said plastic is selected from the group consisting of: polyetheretherketone, carbon fiber polyetheretherketone, polyethelyene, polytetrafluoroethylene, and polyphenylsulfone.

9. The provisional prosthetic component of claim 7, wherein said core material is a metal.

10. The provisional prosthetic component of claim 1, wherein said first portion is overmolded onto said core.

11. A provisional prosthetic component system, comprising:
a body sized to replicate an orientation of at least one component of a modular prosthetic component, said body comprising:
a core formed of a core material; and
a first portion overlying said core, said first portion having a provisional prosthetic junction surface sized and shaped to mate with a final modular prosthetic junction surface of a component of said modular prosthetic component, said provisional prosthetic junction formed from a plastic, the final modular prosthetic junction surface formed from a final modular prosthetic material having a higher scratch hardness than said plastic from which the provisional prosthetic junction surface is formed, whereby interaction of said plastic with the final modular prosthetic material will more likely cause scratching of said plastic than the final modular prosthetic material.

12. The provisional prosthetic component of claim 11, wherein said core material is stiffer than said provisional prosthetic material from which said provisional prosthetic junction surface is formed.

13. The provisional prosthetic component system of claim 11, wherein said core includes a projection and said first portion includes a cavity sized for receipt of said projection to secure said first portion to said core.

14. The provisional prosthetic component system of claim 11, wherein said plastic is selected from the group consisting of: polyetheretherketone, carbon fiber polyetheretherketone, polyethelyene, polytetrafluoroethylene, and polyphenylsulfone.

15. The provisional prosthetic component system of claim 11, wherein said core material is a metal.

16. The provisional prosthetic component system of claim 11, wherein the scratch hardness of the final modular prosthetic material is substantially greater than the scratch hardness of said plastic from which the provisional prosthetic junction surface is formed.

17. A method for trialing a prosthetic device comprising the steps of:
implanting a component of a modular prosthesis within a joint of a patient's body;
attaching a provisional component to the component of the modular prosthesis, the provisional component having a body sized to replicate an orientation of at least one component of the modular prosthetic component, the body comprising:
a core formed of a core material; and
a first portion overlying the core, the first portion having a provisional prosthetic junction surface sized and shaped to mate with a final modular prosthetic junction surface of the component of a modular prosthesis, the provisional prosthetic junction formed from a provisional material, the final modular prosthetic junction surface formed from a final modular prosthetic material, the provisional prosthetic material having a lower scratch hardness than the final modular prosthetic material, whereby interaction of the provisional prosthetic material with the final modular prosthetic material will more likely cause scratching of the provisional prosthetic material than the final modular prosthetic material, the core material having a stiffness greater than the provisional prosthetic material from which the provisional prosthetic junction surface is formed;
reducing the joint, including the provisional component;
evaluating the performance of the provisional component; and
replacing the provisional component with a corresponding component of the modular prosthesis.

18. The method of claim 17, further comprising, between the evaluating step and the replacing step, the steps of:
removing the core from the first portion of the provisional component;
attaching another core to the first portion of the provisional component; and
evaluating the performance of the provisional component.

19. The method of claim 17, wherein the step of evaluating comprises: conducting range of motion testing.

20. The method of claim 17, further comprising, between the evaluating step and the replacing step, the steps of:
(a) removing the provisional component from the component of the modular prosthesis;
(b) attaching another provisional component to the component of the modular prosthesis;
(c) evaluating the performance of the provisional component;
(d) repeating steps (a) through (c) until the performance of the provisional component as determined in step (c) is satisfactory.
